SEQUENCE LISTING (30 sequences)

SEQ ID NO: 1 - protein, 7 aa, Artificial Sequence
  Synthetic Peptide
IDDYTRA

SEQ ID NO: 2 - protein, 7 aa, Artificial Sequence
  Synthetic Peptide
HPPLHHA

SEQ ID NO: 3 - protein, 7 aa, Artificial Sequence
  Synthetic Peptide
SPSTHWK

SEQ ID NO: 4 - protein, 7 aa, Artificial Sequence
  Synthetic Peptide
GSPATAA

SEQ ID NO: 5 - protein, 7 aa, Artificial Sequence
  Synthetic Peptide
GKVQAQS

SEQ ID NO: 6 - protein, 7 aa, Artificial Sequence
  Synthetic Peptide
YPVTPSI

SEQ ID NO: 7 - protein, 7 aa, Artificial Sequence
  Synthetic Peptide
IPTLPSS

SEQ ID NO: 8 - protein, 7 aa, Artificial Sequence
  Synthetic Peptide
YQGASEN

SEQ ID NO: 9 - protein, 7 aa, Artificial Sequence
  Synthetic Peptide
EHITTSN

SEQ ID NO: 10 - protein, 7 aa, Artificial Sequence
  Synthetic Peptide
RILITIP

SEQ ID NO: 11 - protein, 7 aa, Artificial Sequence
  Synthetic Peptide
IPITNLR

SEQ ID NO: 12 - protein, 7 aa, Artificial Sequence
  Synthetic Peptide
TTSTRHI

SEQ ID NO: 13 - protein, 7 aa, Artificial Sequence
  Synthetic Peptide
NERALTL

SEQ ID NO: 14 - protein, 7 aa, Artificial Sequence
  Synthetic Peptide
MQTVEVT

SEQ ID NO: 15 - protein, 7 aa, Artificial Sequence
  Synthetic Peptide
SWGTQTD

SEQ ID NO: 16 - protein, 7 aa, Artificial Sequence
  Synthetic Peptide
TLPASSV

SEQ ID NO: 17 - protein, 135 aa, Artificial Sequence
  Synthetic Peptide
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 18 - protein, 157 aa, Artificial Sequence
  Synthetic Peptide
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 19 - protein, 112 aa, Artificial Sequence
  Synthetic Peptide
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 20 - protein, 119 aa, Artificial Sequence
  Synthetic Peptide
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 21 - protein, 136 aa, Artificial Sequence
  Synthetic Peptide
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 22 - protein, 161 aa, Artificial Sequence
  Synthetic Peptide
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 23 - protein, 155 aa, Artificial Sequence
  Synthetic Peptide
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 24 - protein, 294 aa, Artificial Sequence
  Synthetic Peptide
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 25 - protein, 197 aa, Artificial Sequence
  Synthetic Peptide
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 26 - protein, 443 aa, Artificial Sequence
  Synthetic Peptide
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 27 - protein, 465 aa, Artificial Sequence
  Synthetic Peptide
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 28 - protein, 178 aa, Zoarces americanus
MAEQSDKDVKYYTLEEIQKHKDSKSTWVILHHKVYDLTKFLEEHPGGEEVLGEQAGGDATENFEDVGHSTDARELSKTYIIGELHPDDRSKIAKPSETLDDDDKHMNQASVVANQLIPINTALTLVMMRSEVVTPVGIPAEDIPRLVSMQVNRAVPLGTTLMPDMVKGYPPAHHHHHH

SEQ ID NO: 29 - protein, 97 aa, Choristoneura fumiferana
MDGSCTNTNSQLSANSKCEKSTLTNCYVDKSEVYGTTCTGSRFDGVTITTSTSTGSRISGPGCKISTCIITGGVPAPSAACKISGCTFSANHHHHHH

SEQ ID NO: 30 - protein, 317 aa, Lolium perenne
MTTQSPLNSFYATGTAQAVSEPIDVESHLGSITPAAGAQGSDDIGYAIVWIKDQVNDVKLKVTLRNAEQLKPYFKYLQIQITSGYETNSTALGNFSETKAVISLDNPSAVIVLDKEDIAVLYPDKTGYTNTSIWVPGEPDKIIVYNETKPVAILNFKAFYEAKEGMLFDSLPVIFNFQVLQVGGGSGGDPGGMDEQPNTISGSNNTVRSGSKNVLAGNDNTVISGDNNSVSGSNNTVVSGNDNTVTGSNHVVSGTNHIVTDNNNNVSGNDNNVSGSFHTVSGGHNTVSGSNNTVSGSNHVVSGSNKVVTDAHHHHHH

(19) United States
(12) Patent Application Publication
(10) Pub. No.: US 2019/0201721 A1
(43) Pub. Date: Jul. 4, 2019

(54) ORAL CARE PRODUCTS AND METHODS COMPRISING HYDROXYAPATITE BINDING PROTEINS

(71) Applicant: BASF SE, Ludwigshafen am Rhein (DE)

(72) Inventors: Nina Schneider, Edingen-Neckarhausen (DE); Thomas Subkowski, Chriesheim (DE); Stefan Jenewein, Neustadt (DE); Marvin Karos, Plankstadt (DE); Claus Bollschweiler, Heidelberg (DE); Volker Wendel, Seeheim-Jugenheim (DE); Thorsten Henkes, Nürtingen (DE); Sandra Facey, Leinfelden-Echterdingen (DE); Bernhard Hauer, Fußgönheim (DE)

(21) Appl. No.: 15/771,236

(22) PCT Filed: Oct. 25, 2016
(86) PCT No.: PCT/EP2016/075651

§ 371 Date: Apr. 26, 2018

Related U.S. Application Data
(60) Provisional application No. 62/246,165, filed on Oct. 26, 2015.

(51) Int. Cl. A61Q 11/00; A61K 8/64

(52) U.S. Cl. CPC A61Q 11/00; A61K 8/24; A61K 8/64

(57) ABSTRACT

Provided herein are oral care compositions comprising HABP, which are useful in methods of repairing or inhibiting dental erosion, promoting dental remineralization, and/or enhancing the anti-cavity effects of fluoride.

Dental enamel is a thin, hard layer of calcified material that covers the crown of teeth. The major mineral component of dental enamel is hydroxyapatite, a crystalline form of calcium phosphate. Chemical erosion of dental enamel may arise from tooth exposure to acidic food and drinks or to stomach acids arising from gastric reflux. The erosion of dental enamel can lead to enhanced tooth sensitivity due to increased exposure of the dentin tubules and increased dentin visibility leading to the appearance of more yellow teeth. The salivary pellicle (a thin layer of salivary glycoproteins deposited on teeth) is integral in protecting the teeth against an erosive challenge. As a result, people that experience xerostomia are more susceptible to acid erosion damage.

Existing methods developed to help prevent enamel erosion include incorporating a source of free fluoride into oral care compositions. Fluoride reduces damage to the enamel, through the formation of fluorapatite, which dissolves at a lower pH than hydroxyapatite and so is more resistant to acid damage. Stannous salts have also been incorporated into dentifrice formulations to protect the enamel surface similarly, by forming a more acid resistant mineral layer. Polymers have also been described that coat and protect the enamel surface.

Acids are also generated in the oral cavity when plaque containing cariogenic bacteria metabolize carbohydrates. Since plaque forms a barrier controlling the kinetics of proton and mineral diffusion through the enamel, plaque acids cause carious lesions. Incorporating fluoride ions in dentifrice formulations is the most common method to mitigate the effects of plaque acids. Fluoride reduces the rate of demineralization and enhances remineralization. Several approaches have also been developed to stabilize calcium phosphate salts or control the plaque pH to enhance remineralization.

Although methods have been developed to mitigate the effects of non-bacteria and bacteria generated acids on the teeth, there is still the need to provide improved oral care compositions that effectively repair the enamel from the effects of acid erosion and bacteria acids.

### BRIEF SUMMARY

The present inventors have unexpectedly found that Hydroxyapatite Binding Polypeptides (HABPs) are effective in repairing or mitigating the effects of dental erosion, promoting dental remineralization, and enhancing the anti-cavity effects of fluoride.

For example, in one embodiment, HABPs are prepared for formulation with ingredients of a suitable orally acceptable carrier, by diluting in buffer, e.g., a phospate buffer such as Na2HPO4 buffer (1.5 mM) and CaCl₂) (2.5 mM), to provide a buffered solution having approximately neutral or slightly basic pH, e.g., pH 7-8, e.g., about pH 7.5, filtering and centrifuging the solution to obtain a filtrate comprising the HABP. A biocide (for example cetylpyridinium chloride at 0.1%) and fluoride may be added to the filtrate. The HABP may then be combined with components of an orally acceptable carrier, for example a toothpaste or mouthwash base, to provide an oral care composition for repairing or mitigating the effects of dental erosion, promoting dental remineralization, and enhancing the anti-cavity effects of fluoride.

This disclosure thus relates to an oral care composition (Composition 1), for example a dentifrice, comprising:
a) HABP;
b) an orally acceptable carrier,
   wherein the HABP is present in the composition in an amount of from 0.01 weight % to 3 weight % by total weight of the composition. For example, the disclosure provides:
1.1. Composition 1 wherein the HABP has a polypeptide sequence SEQ ID NO:1, 2, 3, 4.
1.2. Any foregoing Composition comprising fluoride.
1.3. Any foregoing Composition wherein the HABP has been neutralized to approximately neutral or slightly basic pH, e.g., pH 7-8, e.g., using a phosphate buffer.
1.4. Any foregoing Composition wherein the HABP comprises a biocide, e.g., cetylpyridinium chloride (CPC) at an effective concentration, e.g., 0.1% by weight of the filtrate.
1.5. Any foregoing Composition wherein the HABP is present in the composition in an amount of from 0.1 weight % to 3 weight % by total weight of the composition, e.g., 0.2 weight % to 2 weight %, e.g., about 0.2 weight %, about 1 weight %, about 1.5 weight % or about 2 weight percent by total weight of the composition.
1.6. Any foregoing composition wherein the Composition comprises an effective amount of fluoride.
1.7. Any foregoing Composition which comprises an amount of fluoride of 100 ppm to 2500 ppm, for example, 250 ppm to 750 ppm, for example about 500 ppm fluoride.
1.8. Any foregoing Composition comprising an orally acceptable zinc salt or oxide, for example selected from zinc oxide, zinc citrate, zinc lactate, zinc phosphate, zinc acetate, zinc chloride, zinc complexes with amino acids, and mixtures of any of the foregoing, for example wherein the amount of zinc is from 0.1 weight % to 3 weight %, e.g., about 1 to about 2 weight %, calculated by weight of zinc ion.
1.9. Any foregoing Composition comprising an orally acceptable stannous salt, for example SnF₂ or SnCl₂.
1.10. Any foregoing Composition wherein the composition is in a form selected from a mouthrinse, a toothpaste, a tooth gel, a tooth powder, a non-abrasive gel, a mousse, a foam, a mouth spray, and a tablet, for example dentifrice, e.g., a toothpaste or mouthrinse.
1.11. Any foregoing Composition, wherein the composition further comprises one or more agents selected from: abrasives, pH modifying agents, surfactants, foam modulators, thickening agents, viscosity modifiers, humectants, anti-calculus or tartar control agents, sweeteners, flavorants and colorants.
1.12. Any foregoing Composition wherein the composition is a toothpaste.
1.13. Any foregoing Composition comprising one or more soluble phosphate salts, for example wherein by “soluble phosphate salts” is meant an orally acceptable phosphate salt having a solubility in water of at least 1 g/100 ml at 25° C.; for example wherein the one or more soluble phosphate salts are sodium and/or potassium salts of pyrophosphates and/or polyphosphates, e.g., tripolyphosphates; e.g., wherein the one or more soluble phosphate salts comprise tetrasodium pyrophosphate (TSPP), sodium tripolyphosphate (STPP) or a combination of TSPP and STPP; for example, wherein the one or more soluble phosphate salts are present in an amount of 1-20%, e.g., 2-8%, e.g., ca. 5%, by weight of the composition.
1.14. Any foregoing Composition wherein the fluoride is provided by a salt selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.15. Any foregoing Composition which is a dentifrice comprising a humectant, e.g., selected from glycerin, sorbitol, propylene glycol, polyethylene glycol, xylitol, and mixtures thereof, e.g. comprising at least 30%, e.g., 40-50% glycerin, by weight of the composition.
1.16. Any foregoing Composition which is a dentifrice comprising one or more surfactants, e.g., selected from anionic, cationic, zwitterionic, and nonionic surfactants, and mixtures thereof, e.g., wherein the dentifrice base comprises an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS) by weight of the composition.
1.17. Any foregoing Composition which is a dentifrice comprising a zwitterionic surfactant, for example a betaine surfactant, for example cocamidopropylbetaine, e.g. in an amount of from about 0.1% to about 4.5% by weight, e.g. 0.5-2% cocamidopropylbetaine by weight of the composition.
1.18. Any foregoing Composition which is a dentifrice comprising a viscosity modifying amount of one or more of polysaccharide gums, for example xanthan gum or carrageenan, silica thickener, and combinations thereof.
1.19. Any foregoing Composition which is a dentifrice comprising gum strips or fragments.
1.20. Any foregoing Composition comprising flavoring, fragrance and/or coloring.
1.21. Any foregoing Composition which is a dentifrice comprising an effective amount of one or more antibacterial agents, for example comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing; e.g., comprising triclosan or cetylpyridinium chloride.
1.22. Any foregoing Composition which is a dentifrice comprising a whitening agent, e.g., a selected from the group consisting of peroxides, metal chlorites, perborates, percarbonates, peroxyacids, hypochlorites, and combinations thereof; e.g. hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate).
1.23. Any foregoing Composition which is a dentifrice comprising an agent that interferes with or prevents bacterial attachment, e.g., solbrol or chitosan.
1.24. Any foregoing Composition which is a dentifrice comprising a soluble calcium salt, e.g., selected from calcium sulfate, calcium chloride, calcium nitrate, calcium acetate, calcium lactate, and combinations thereof.
1.25. Any foregoing Composition which is a dentifrice comprising a physiologically or orally acceptable potassium salt, e.g., potassium nitrate or potassium chloride, in an amount effective to reduce dentinal sensitivity.
1.26. Any foregoing Composition which is a dentifrice comprising an anionic polymer, e.g., a synthetic anionic polymeric polycarboxylate, e.g., wherein the anionic polymer is selected from 1:4 to 4:1 copolymers of maleic anhydride or acid with another polymerizable ethylenically unsaturated monomer; e.g., wherein the anionic polymer is a methyl vinyl ether/maleic anhydride (PVM/MA) copolymer having an average molecular weight (M.W.) of about 30,000 to about 1,000,000, e.g. about 300,000 to about 800,000, e.g., wherein the anionic polymer is about 1-5%, e.g., about 2%, of the weight of the composition.
1.27. Any foregoing Composition which is a dentifrice comprising a breath freshener, fragrance or flavoring.
1.28. Any of the foregoing Compositions, wherein the pH of the composition is approximately neutral, e.g., about pH 7.
1.29. Any of the forgoing compositions for use to reduce and inhibit acid erosion, clean the teeth, reduce bacterially-generated biofilm and plaque, reduce gingivitis, inhibit tooth decay and formation of cavities, and reduce dentinal hypersensitivity.
1.30. Any foregoing Composition for use in reducing, inhibiting or repairing dental enamel erosion.
1.31. Any foregoing composition for use in promoting remineralization of dental enamel.
1.32. Any foregoing composition for use in enhancing the anti-cavity effects of fluoride.

A particular novel embodiment of Composition 1 is a dentifrice comprising
a) An HABP; for example comprising dry substance of at least 20.0%, total nitrogen of at least 2.5%, pH value of 4.0-5.0, and ash content 1.0% or less; wherein the partially hydrolyzed wheat protein has been neutralized to approximately neutral or slightly basic pH;
b) optionally an effective amount of fluoride;
c) an orally acceptable carrier,
   for example wherein the amount of HABP is 0.1 weight % to 2 weight and
   for example wherein fluoride is present in an amount of 100 ppm to 1000 ppm, for example, about 500 ppm.

In one aspect, the disclosure provides any of Compositions 1, et seq. for use in repairing or inhibiting dental erosion, promoting remineralization, and/or enhancing the anti-cavity effects of fluoride; for example for use in any of the following methods according to Method 1, et seq.

In another aspect, the disclosure provides a method (Method 1) of repairing or inhibiting dental erosion, promoting dental remineralization, and/or enhancing the anti-cavity effects of fluoride comprising applying to the teeth a composition, e.g., any of Composition 1, et seq. for example an oral care composition comprising:
a) HABP
b) an orally acceptable carrier,
   wherein the HABP is present in the composition in an amount of from 0.01 weight % to 3 weight % by total weight of the composition. For example, the disclosure provides:
1.1. Method 1 wherein the HABP has the polypeptide sequence SEQ ID NO:1,2,3,4
1.2. Any foregoing Method wherein the HABP comprises fluoride.
1.3. Any foregoing Method wherein the HABP has been neutralized to approximately neutral or slightly basic pH, e.g., pH 7-8, e.g., using a phosphate buffer.
1.4. Any foregoing Method wherein the HABP comprises a biocide, e.g., cetylpyridinium chloride (CPC) at an effective concentration, e.g., 0.1% by weight of the filtrate.
1.5. Any foregoing Method wherein the HABP is present in the composition in an amount of from 0.1 weight % to 3 weight % by total weight of the composition, e.g., 0.2 weight % to 2 weight %, e.g., about 0.2 weight %, about 1 weight %, about 1.5 weight % or about 2 weight percent by total weight of the composition.
1.6. Any foregoing composition wherein the Composition comprises an effective amount of fluoride.
1.7. Any foregoing Method wherein the amount of fluoride is 100 ppm to 1000 ppm, for example, about 500 ppm fluoride.
1.8. Any foregoing Method wherein the composition is in a form selected from a mouthrinse, a toothpaste, a tooth gel, a tooth powder, a non-abrasive gel, a mousse, a foam, a mouth spray, and a tablet.
1.9. Any foregoing Method, wherein the composition further comprises one or more agents selected from: abrasives, pH modifying agents, surfactants, foam modulators, thickening agents, viscosity modifiers, humectants, anti-calculus or tartar control agents, sweeteners, flavorants and colorants.
1.10. Any foregoing Method wherein the composition is a dentifrice, e.g., a toothpaste.
1.11. Any foregoing Method wherein the composition is selected from any of Compositions 1, et seq., supra.
1.12. Any foregoing Method which is a method for reducing, inhibiting or repairing dental erosion, for example erosion of the enamel, for example wherein the composition is applied to the teeth of a patient having been identified as having dental erosion or being at elevated risk of having dental erosion.
1.13. Any foregoing Method which is a method for promoting dental remineralization, for example remineralization of the enamel, for example wherein the composition is applied to the teeth of a patient having been identified as having demineralization.
1.14. Any foregoing Method which is a method for enhancing the anti-cavity effects of fluoride, for example wherein the composition is applied to the teeth of a patient having been identified as having early signs of tooth decay, for example early enamel caries, or as having active decay, or as being at elevated risk of tooth decay.
1.15. Method 1.15 wherein the composition comprises an effective amount of fluoride and/or wherein the method further comprises administration of an oral care product comprising an effective amount of fluoride mouth rinse or toothpaste comprising an effective amount of fluoride.

In another embodiment, the disclosure provides the use of an HABP in the manufacture of an oral care composition, for example according to any of Compositions 1, et seq. for repairing or inhibiting dental erosion, promoting remineralization, and/or enhancing the anti-cavity effects of fluoride, e.g., in any of Methods 1, et seq.

In another aspect, the disclosure provides a method (Method 2) of making an oral care product, e.g. an oral care product useful for repairing or inhibiting dental erosion, promoting dental remineralization, and/or enhancing the anti-cavity effects of fluoride, e.g., a product according to any of Composition 1, et seq., comprising
a) neutralizing an HABP by dilution with an aqueous buffer solution, e.g., a phosphate buffer solution, to obtain a solution having approximately neutral or slightly basic pH, e.g., pH 7-8;
b) filtering and centrifuging the solution product of a) to obtain a filtrate comprising the HABP;
c) adding fluoride (e.g., in the form of an orally acceptable salt containing fluoride, for example sodium fluoride or sodium monofluorophosphate), and optionally a biocide (e.g., cetylpyridinium chloride at an effective amount, e.g., 0.01 to 1%, e.g., about 0.1% by weight of the filtrate) to the filtrate product of b);
d) admixing the product of c) to components of an orally acceptable carrier to obtain an oral care composition comprising the HABP in an amount of from 0.01 weight % to 3 weight % by total weight of the composition.

For example, the disclosure provides an oral care composition comprising HABP, e.g., a composition according to any of Composition 1, et seq., wherein the oral care composition is obtained or obtainable by the process of Method 2, et seq.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Hydroxyapatite Binding Polypeptides (HABPs)

Hydroxyapatite Binding Polypeptides (HABPs) according to this invention are polypeptides which have been designed for a specific binding to Hydroxyapatite which is the main component of dental enamel as well as for promoting dental remineralization, and for enhancing the anti-cavity effects of fluoride.

1. The HABPs can be described by the general Formula (I)

a₀₋₁-b₀₋₁₀-y₁₋₁₀-c₀₋₁₀-d₀₋₁ (I)
wherein
a,d stand independently from each other for a hydrophobin or an antifreeze protein sequence or an oral care active ingredient, e.g. cooling compound, anti-microbial compound, whitening compound
b,c stand for independently from each other for an amino acid
y stands for a polypeptide sequence depicted in SEQ ID NO: 1 until SEQ ID NO:16.

The HABP have a core sequence (“y”) which is polypeptide elected from the group of SEQ ID NO: 1, 2, 3, 4, 5, 6, 7, 8, 9, 10, 11, 12, 13, 14, 15, 16. Preferred sequences y are SEQ ID NO:1,2,3,4. The HABP can have one, two, three, four, five, six, seven, eight, nine or ten y sequences. In case of multiple y-sequences the y can be identical (“homomer”) or different (“heteromer”), or partly identical and partly different. For example y₃ can stand for the polypeptide SEQ ID NO:1-SEQ ID NO:3-SEQ ID NO:11, i.e. 21 amino acids.

The HABP can have at the amino- and/or at the carboxyterminal end of y additional amino acids b₀₋₁₀ and c₀₋₁₀. “b” and “c” stand for any of the 20 natural amino acids (Ala, Cys, Asp, Glu, Phe, Gly, His, Ile, Lys, Leu, Met, Asn, Pro, Gln, Arg, Ser, Thr, Val, Trp, Tyr) and the indices 0-10 stand for no (0), one (1), two (2), three (3) . . . up to ten (10) amino acids. In case of e.g. four amino acids these four amino acids can be identical or different. For example b₄ can stand for Ala-Gly-Ser-Ser. The sequences “b” and “c” are spacer or additional sequences, which are created sometimes by cloning the respective gene fragments (recognition sites for restriction enzymes or primer) and which have usually no specific effect for the hydroxyapatite binding or the dental remineralization enhancement. The sequences “b” and “c” could if necessary contain a cleavage side for proteases which occur in the oral cavity.

Preferred amino acids for b and c are uncharged amino acids with non-bulky side chains such as Gly, Ala, Ser, Thr.

Furthermore the HABP can comprise in addition to “y” and—if wished “b” and/or “c” further polypeptides “a” and/or “d” which can be a hydrophobin polypeptide and/or an antifreeze-protein (AFP) or an oral care active ingredient. For example one embodiment of an HABP can have a hydrophobin polypeptide fused to a y sequence followed by another hydrophobin. Another embodiment of an HABP can be an AFP polypeptide (“a”) fused to a y sequence fused to another y sequence followed by an oral care active ingredient molecule.

Hydrophobin means a well-defined class of proteins (Wessels, 1997, Adv. Microb. Physio. 38: 1-45; Wosten, 2001, Annu Rev. Microbiol. 55: 625-646) capable of self-assembly at a hydrophobic/hydrophilic interface, and having a conserved sequence:

Xₙ—C—X₅₋₉—C—C—X₁₁₋₃₉—C—X₈₋₂₃—C—X₅₋₉—C—C—X₆₋₁₈—C—Xₘ

where X represents any amino acid, and n and m independently represent an integer. Typically, a hydrophobin has a length of up to 125 amino acids. The cysteine residues (C) in the conserved sequence are part of disulphide bridges. In the context of this invention, the term hydrophobin has a wider meaning to include functionally equivalent proteins still displaying the characteristic of self-assembly at a hydrophobic-hydrophilic interface resulting in a protein film, or parts thereof still displaying the characteristic of self-assembly at a hydrophobic-hydrophilic interface resulting in a protein film. In accordance with the definition of this invention, self-assembly can be detected by adsorbing the protein to Teflon and using Circular Dichroism to establish the presence of a secondary structure (in general, a-helix) (De Vocht et al., 1998, Biophys. J. 74: 2059-68). The formation of a film can be established by incubating a Teflon sheet in the protein solution followed by at least three washes with water or buffer (Wosten et al., 1994, Embo. J. 13: 5848-54). The protein film can be visualised by any suitable method, such as labelling with a fluorescent marker or by the use of fluorescent antibodies, as is well established in the art. m and n typically have values ranging from 0 to 2000, but more usually m and n in total are less than 200 or 300. The definition of hydrophobin in the context of this invention includes fusion proteins of a hydrophobin and another polypeptide as well as conjugates hydrophobin and other molecules such as polysaccharides.

Hydrophobins identified to date are generally classed as either class I or class II. Both types have been identified in fungi as secreted proteins that self-assemble at hydrophobic-hydrophilic interfaces into amphipathic films.

The hydrophobins can be obtained by extraction from native sources, such as filamentous fungi, by any suitable process. For example, hydrophobins can be obtained by culturing filamentous fungi that secrete the hydrophobin into the growth medium or by extraction from fungal mycelia with 60% ethanol. It is particularly preferred to isolate hydrophobins from host organisms that naturally secrete hydrophobins. Preferred hosts are hyphomycetes (e.g. Trichoderma), basidiomycetes and ascomycetes. Particularly preferred hosts are food grade organisms, such as Cryphonectria parasitica which secretes a hydrophobin termed cryparin (Maccabe and Van Alfen, 1999, App. Environ. Microbiol 65: 5431-5435).

Alternatively, hydrophobins can be obtained by the use of recombinant technology. For example host cells, typically micro-organisms, may be modified to express hydrophobins and the hydrophobins can then be isolated and used in accordance with the present invention. Techniques for introducing nucleic acid constructs encoding hydrophobins into host cells are well known in the art. More than 34 genes coding for hydrophobins have been cloned, from over 16 fungal species (see for example WO96/41882 which gives the sequence of hydrophobins identified in Agaricus bisporus; and Wosten, 2001, Annu. Rev. Microbiol. 55: 625-646). Recombinant technology can also be used to modify hydrophobin sequences or synthesise novel hydrophobins having desired/improved properties. Typically, an appropriate host cell or organism is transformed by a nucleic acid construct that encodes the desired hydrophobin. The nucleotide sequence coding for the polypeptide can be inserted into a suitable expression vector encoding the necessary elements for transcription and translation and in such a manner that they will be expressed under appropriate conditions (e.g. in proper orientation and correct reading frame and with appropriate targeting and expression sequences). The methods required to construct these expression vectors are well known to those skilled in the art.

A number of expression systems may be used to express the polypeptide coding sequence. These include, but are not limited to, bacteria, fungi (including yeast), insect cell systems, plant cell culture systems and plants all transformed with the appropriate expression vectors. Preferred hosts are those that are considered food grade—‘generally regarded as safe’ (GRAS).

Suitable fungal species, include yeasts such as (but not limited to) those of the genera Saccharomyces, Kluyveromyces, Pichia, Hansenula, Candida, Schizo saccharomyces and the like, and filamentous species such as (but not limited to) those of the genera Aspergillus, Trichoderma, Mucor, Neurospora, Fusarium and the like.

The sequences encoding the hydrophobins are preferably at least 80% identical at the amino acid level to a hydrophobin identified in nature, more preferably at least 95% or 100% identical. However, persons skilled in the art may make conservative substitutions or other amino acid changes that do not reduce the biological activity of the hydrophobin. For the purpose of the invention these hydrophobins possessing this high level of identity to a hydrophobin that naturally occurs are also embraced within the term “hydrophobins”.

Hydrophobins can be purified from culture media or cellular extracts by, for example, the procedure described in WO01/57076 which involves adsorbing the hydrophobin present in a hydrophobin-containing solution to surface and then contacting the surface with a surfactant, such as Tween 20, to elute the hydrophobin from the surface. See also Collen et al., 2002, Biochim Biophys Acta. 1569: 139-50; Calonje et al., 2002, Can. J. Microbiol. 48: 1030-4; Askolin et al., 2001, Appl Microbiol Biotechnol. 57: 124-30; and De Vries et al., 1999, Eur J Biochem. 262: 377-85. Typically, the hydrophobin is in an isolated form, typically at least partially purified, such as at least 10% pure, based on weight of solids. By “isolated form”, we mean that the hydrophobin is not added as part of a naturally-occurring organism, such as a mushroom, which naturally expresses hydrophobins. Instead, the hydrophobin will typically either have been extracted from a naturally-occurring source or obtained by recombinant expression in a host organism.

Hydrophobin proteins can be divided into two classes: Class I, which are largely insoluble in water, and Class II, which are readily soluble in water.

Preferably, the hydrophobins chosen are Class I hydrophobins. More preferably the hydrophobins used are Class I hydrophobins such as DewA, RodA. The hydrophobin can be from a single source or a plurality of sources e.g. a mixture of two or more different hydrophobins. The total amount of hydrophobin in compositions of the invention will generally be at least 0.001%, more preferably at least 0.005 or 0.01%, and generally no greater than 2% by total weight hydrophobin based on the total weight of the composition.

Hydrophobins in the definition used in this invention comprise also fusion proteins of a hydrophobin and another protein. These can be described by the general formula (II)

Xₙ—C¹—X₁₋₅₀—C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (II)

where X may be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly) and the indices at X indicate the number of amino acids, with the indices n and m being numbers between 0 and 500, preferably between 15 and 300, and C being cystein, with the proviso that at least one of the peptide sequences abbreviated as Xₙ or Xₘ is a peptide sequence of at least 20 amino acids in length which is not linked to a hydrophobin naturally, which polypeptides change the contact angle by at least 20° after coating of a glass surface.

The cysteins designated by C¹ to C⁸ may either be in the reduced form or form disulfide bridges with one another in the proteins of the invention. Particular preference is given to the intramolecular formation of C—C bridges, in particular those having at least one, preferably 2, particularly preferably 3, and very particularly preferably 4, intramolecular disulfide bridges selected from the following group: C¹ with C²; C³ with C⁴, C⁵ with C⁶, C⁷ with C⁸. If cysteins are also used in the positions designated by X, the numbering of the individual cystein positions in the general formulae may change accordingly.

Particularly advantageous polypeptides are those of the general formula (III)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (III)

where X may be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly) and the indices at X indicate the number of amino acids, with the indices n and m being numbers between 2 and 300 and C being cystein, with the proviso that at least one of the peptide sequences abbreviated as Xₙ or Xₘ is a peptide sequence of at least 35 amino acids in length which is not linked to a hydrophobin naturally, which polypeptides change the contact angle by at least 20° after coating of a glass surface.

Very particularly advantageous are those polypeptides of the general formula (IV)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (IV)

where X may be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly) and the indices at X indicate the number of amino acids, with the indices n and m being numbers between 0 and 200 and C being cystein, with the proviso that at least one of the peptide sequences abbreviated as Xₙ or Xₘ is a peptide sequence of at least 40 amino acids in length which is not linked to a hydrophobin naturally, which polypeptides change the contact angle by at least 20° after coating of a glass surface.

Preferred embodiments of the described invention are polypeptides having the general structural formula (I), (II) or (III), this structural formula comprising at least one Class I hydrophobin, preferably at least one dewA, rodA, hypA, hypB, sc3, basf1, basf2, hydrophobin, or parts or derivatives thereof. Said hydrophobins are structurally characterized in the sequence listing below. It is also possible for a plurality, preferably 2 or 3, structurally identical or different hydrophobins to be linked to one another and to a corresponding suitable polypeptide sequence which is not connected with a hydrophobin naturally.

Particularly preferred embodiments of the present invention are the hydrophobin proteins having the polypeptide sequences depicted in SEQ ID NO:17 (dewA), SEQ ID NO:18 (rodA), SEQ ID NO:19 (HypA), SEQ ID NO:20 (HypB), SEQ ID NO:21 (Sc3), SEQ ID NO:22 (BASF1), SEQ ID NO:23 (BASF2).

The proteins of the invention have in at least one position abbreviated by Xₙ or Xₘ a polypeptide sequence comprising at least 20, preferably at least 35, particularly preferably at least 50, and in particular at least 100, amino acids (also referred to as fusion partner herein below), which is not linked naturally to a hydrophobin. This is intended to express the fact that the proteins of the invention consist of a hydrophobin moiety and a fusion partner moiety which do not occur together in this form in nature.

The fusion partner moiety may be selected from a multiplicity of proteins. It is also possible to link a plurality of fusion partners to one hydrophobin moiety, for example at the amino terminus (Xₙ) and at the carboxy terminus (Xₘ) of the hydrophobin moiety. However, it is also possible to link, for example, two fusion partners to a single position (Xₙ or Xₘ) of the protein of the invention.

Particularly suitable fusion partners are polypeptides which occur naturally in microorganisms, in particular in E. coli or Bacillus subtilis. Examples of such fusion partners are the sequences yaad (SEQ ID NO:24), yaae (SEQ ID NO: 25) resulting in new hydrophobin fusion proteins, e.g. SEQ ID NO:26 and NO:27. Another preferred fusion partner is thioredoxin. Very useful are also fragments or derivatives of said sequences which comprise only part, preferably 70-99%, particularly preferably 80-98%, of said sequences or in which individual amino acids or nucleotides have been altered in comparison with said sequence. For example, additional amino acids, in particular two additional amino acids, preferably the amino acids Arg, Ser, may be attached to the C-terminal end of the yaad and yaae sequences. It is also possible with preference for additional amino acids, for example amino acid No. 2 (Gly) in, to be inserted in the yaae or yaad sequence compared to the naturally occurring sequence.

AFP means Antifreeze Proteins (AFPs) which are proteins from organisms such as certain vertebrates, plants, fungi and bacteria that permit their survival in subzero environments. AFPs are also called Ice structuring proteins. AFPs bind to small ice crystals to inhibit growth and recrystallization of ice that would otherwise be fatal for the organisms. The common feature of AFPS is a thermal hysteresis, which is a difference between the melting point and the freezing point. The addition of AFPS at the interface between solid ice and liquid water inhibits the thermodynamically favored growth of the ice crystal. Ice growth is kinetically inhibited by the AFPs covering the water-accessible surfaces of ice. Thermal hysteresis is easily measured in the lab with a nanolitre osmometer.

There is no common consensus sequence of the AFPs from the different organisms Crit Rev Biotechnol. 2008; 28: 57-82., Properties, potentials, and prospects of antifreeze proteins, Venketesh S1, Dayananda C.; Journal of Experimental Biology 2015; 218: 1846-1855., Animal ice-binding (antifreeze) proteins and glycolipids: an overview with emphasis on physiological function, John G Duman.

Useful for the instant invention are AFPs from fish such as e.g. macrozoarces americanus or from plants such as lolium perenne. Preferred AFPs are such from insects. There are two known types of insect AFPs, Tenebrio and Dendroides AFPs which are both in different insect families. They are similar to one another, and consist of varying numbers of 12 or 13-mer repeats of approximately 8.3-12.5 kD. Especially preferred AFPs from insects are those from choristoneura fumiferana.

Especially preferred AFPs are those with the following polypeptide sequence:

### BM 82 (SEQ ID NO:28),

### BM 80 (SEQ ID NO:29),

### BM 83 (SEQ ID NO:30).

AFP in the sense of this invention can also mean that multiple copies (i.e. multimers) of an AFP are fused together, e.g. two or three AFP—having the identical structure (i.e. homomers) or a different structure (i.e. heteromers)—form a “super AFP”. E.g. an AFP from fish can be fused together with an AFP from insects or two identical fish AFP are fused together.

Furthermore the HABP can comprise in addition to “y” and—if wished “b” and/or “c” further active ingredients “a” or “d” which can be oral care active ingredients.

Oral care active ingredient means compounds, especially low molecular weight compounds which could have beneficial, protective, therapeutic (e.g. chlorhexidine) or cosmetic effect (e.g. Dexpanthenol ((+)-(R)-2,4-Dihydroxy-N-(3-hydroxypropyl)-3,3-dimethylbutyramid), vitamine E) to the teeth or to the oral cavity. Oral care active ingredients comprises substances which have antimicrobial effects in the oral cavity such as triclosan or compounds which effect a cool feeling in the mouth such as menthol or even some stimulating agents like caffeine. Also teeth whitening compounds can be used as oral care active ingredients in the sense of this invention.

Cooling compounds like menthol are known to those skilled in the art and were described e.g. in the patent application US 20150086491 A1. Known examples of cooling compounds include menthol, menthone, N-ethyl-p-menthane carboxamide (WS-3, also known as menthane-3-carboxylic acid-N-ethylamide), N-2,3-trimethyl-2-isopropylbutanamide (WS-23), menthyl lactate (Frescolate® ML), menthone glycerine acetal (Frescolate® MGA), mono-menthyl succinate (Physcool®), mono-menthyl glutarate, 0-menthyl glycerine, and menthyl-N,N-dimethylsuccinamate.

So the HABP according to this invention are polypeptides from a length of seven amino acids up to several hundred amino acids. All HABPs have one or two or 3 or up to 10 y sequences and possibly also some spacer or terminal amino acids b and c and possibly some additional components selected from the group of hydrophobins and/or AFPs and/or oral care active ingredients (e.g. cooling compounds, anti-microbial compounds whitening compounds). The shortest HABPs with just one y sequence already possess the specific hydroxyapatite binding and the dental remineralization property. With the additional sequence a and or d, i.e. with a hydrophobin and/or AFP protein this remineralization property is enhanced respectively additional properties directed to the surface of enamel/dentin.

The HABPs can be manufactured chemically by known methods of peptide synthesis, for example solid phase synthesis according to Merrifield. This method is especially preferred for smaller HABP such as peptides with a chain length of 7 to 18 amino acids.

Particularly useful, however, are genetic methods for manufacturing in which nucleic acid sequences, in particular DNA sequences, coding for the HABP are combined in such a way that gene expression of the combined nucleic acid sequence generates the desired protein in a host organism.

Suitable host organisms (producer organisms) here may be prokaryotes (including Archaea) or eukaryotes, particularly bacteria including halobacteria and methanococci, fungi, insect cells, plant cells and mammalian cells, particularly preferably Escherichia coli, Bacillus subtilis, Bacillus megaterium, Aspergillus oryzea, Aspergillus nidulans, Aspergillus niger, Pichia pastoris, Pseudomonas spec., Lactobacillen, Hansenula polymorpha, Trichoderma reesei, SF9 (or related cells), and others.

The invention moreover relates to expression constructs comprising a nucleic acid sequence coding for a polypeptide of the invention under the genetic control of regulatory nucleic acid sequences, and also vectors comprising at least one of said expression constructs.

Preference is given to such constructs of the invention comprising a promoter 5′ upstream of the particular coding sequence and a terminator sequence 3′ downstream, and also, if appropriate, further customary regulatory elements, in each case operatively linked to said coding sequence.

An “operative linkage” means the sequential arrangement of promoter, coding sequence, terminator and, if appropriate, further regulatory elements in such a way that each of the regulatory elements is able to fulfill its function in accordance with its intended use in connection with expressing the coding sequence.

Examples of sequences which can be operatively linked are targeting sequences and also enhancers, polyadenylation signals and the like. Further regulatory elements comprise selectable markers, amplification signals, origins of replication and the like. Examples of suitable regulatory sequences are described in Goeddel, Gene Expression Technology: Methods in Enzymology 185, Academic Press, San Diego, Calif. (1990).

In addition to these regulatory sequences, the natural regulation of these sequences may still be present upstream of the actual structural genes and, if appropriate, have been genetically modified such that the natural regulation has been switched off and expression of the genes has been increased.

A preferred nucleic acid construct advantageously also comprises one or more of the enhancer sequences already mentioned which are functionally linked to the promoter and enable expression of the nucleic acid sequence to be increased. Additional advantageous sequences such as further regulatory elements or terminators may also be inserted at the 3′ end of the DNA sequences.

The nucleic acids of the invention may be present in the construct in one or more copies. The construct may comprise still further markers such as antibiotic resistances or genes which complement auxotrophies, for selecting for the construct, if appropriate.

Examples of regulatory sequences which are advantageous for the method of the invention are present in promoters such as the cos, tac, trp, tet, trp-tet, lpp, lac, lpp-lac, laclq-T7, T5, T3, gal, trc, ara, rhaP(rhaPBAD) SP6, lambda-PR or lambda-P promoter, which are advantageously used in Gram-negative bacteria. Further examples of advantageous regulatory sequences are present in the Gram-positive promoters amy and SP02, in the yeast or fungal promoters ADC1, MFalpha, AC, P-60, CYC1, GAPDH, TEF, rp28, ADH. It is also possible to use artificial promoters for regulation.

To be expressed in a host organism, the nucleic acid construct is advantageously inserted into a vector such as, for example, a plasmid or a phage, which enables the genes to be expressed optimally in the host. Apart from plasmids and phages, vectors also mean any other vectors known to the skilled worker, i.e., for example, viruses such as SV40, CMV, baculovirus and adenovirus, transposons, IS elements, phasmids, cosmids, and linear or circular DNA and also the Agrobacterium system.

These vectors may either replicate autonomously in the host organism or be replicated chromosomally. These vectors constitute another embodiment of the invention. Examples of suitable plasmids are pLG338, pACYC184, pBR322, pUC18, pUC19, pKC30, pRep4, pHS1, pKK223-3, pDHE19.2, pHS2, pPLc236, pMBL24, pLG200, pUR290, pIN-III″3-B1, tgt11 or pBdCl in E. coli, pIJ101, pIJ364, pIJ702 or pIJ361 in Streptomyces, pUB110, pC194 or pBD214 in Bacillus, pSA77 or pAJ667 in Corynebacterium, pALS1, plL2 or pBB116 in fungi, 2alpha, pAG-1, YEp6, YEp13 or pEMBLYe23 in yeasts or pLGV23, pGHlac+, pBIN19, pAK2004 or pDH51 in plants. Said plasmids are a small selection of the possible plasmids. Further plasmids are well known to the skilled worker and can be found, for example, in the book Cloning Vectors (Eds. Pouwels P. H. et al. Elsevier, Amsterdam-New York-Oxford, 1985, ISBN 0 444 904018).

Advantageously, the nucleic acid construct additionally comprises, for the purpose of expressing the other genes present, also 3′- and/or 5′-terminal regulatory sequences for increasing expression which are selected for optimal expression depending on the host organism and gene or genes selected.

These regulatory sequences are intended to enable the genes and protein expression to be expressed specifically. Depending on the host organism, this may mean, for example, that the gene is expressed or overexpressed only after induction or that it is expressed and/or overexpressed immediately.

In this connection, the regulatory sequences or factors may preferably have a beneficial influence on, and thereby increase, gene expression of the introduced genes. Thus the regulatory elements can advantageously be enhanced at the transcriptional level by using strong transcription signals such as promoters and/or enhancers. Apart from that, however, it is also possible to enhance translation by improving mRNA stability, for example.

In another embodiment of the vector, the vector comprising the nucleic acid construct of the invention or the nucleic acid of the invention may also advantageously be introduced in the form of a linear DNA into the microorganisms and integrated into the genome of the host organism by way of heterologous or homologous recombination. Said linear DNA may consist of a linearized vector such as a plasmid, or only of the nucleic acid construct or the nucleic acid of the invention.

In order to achieve optimal expression of heterologous genes in organisms, it is advantageous to modify the nucleic acid sequences according to the specific codon usage employed in the organism. The codon usage can be readily determined on the basis of computer analyses of other known genes of the organism in question.

An expression cassette of the invention is prepared by fusing a suitable promoter to a suitable coding nucleotide sequence and a terminator signal or polyadenylation signal. For this purpose, use is made of familiar recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which enables the genes to be expressed optimally in the host. Vectors are well known to the skilled worker and can be found, for example, in “Cloning Vectors” (Pouwels P. H. et al., Eds. Elsevier, Amsterdam-New York-Oxford, 1985).

The vectors of the invention can be used to prepare recombinant microorganisms which are transformed, for example, with at least one vector of the invention and may be used for producing the polypeptides of the invention. Advantageously, the above-described recombinant constructs of the invention are introduced into and expressed in a suitable host system. Preference is given here to using common cloning and transfection methods known to the skilled worker, such as, for example, coprecipitation, protoplast fusion, electroporation, retroviral transfection and the like, in order to express said nucleic acids in the particular expression system. Suitable systems are described, for example, in Current Protocols in Molecular Biology, F. Ausubel et al., Eds. Wiley Interscience, New York 1997, or Sambrook et al. Molecular Cloning: A Laboratory Manual, 2nd Ed., Cold Spring Harbor Laboratory, Cold Spring Harbor Laboratory Press, Cold Spring Harbor, N.Y., 1989.

It is also possible according to the invention to prepare homologously recombined microorganisms. For this purpose, a vector is prepared which comprises at least one section of a gene of the invention or of a coding sequence, into which, if appropriate, at least one amino acid deletion, addition or substitution has been introduced in order to modify, for example functionally disrupt, the sequence of the invention (knockout vector). The introduced sequence may, for example, also be a homolog from a related microorganism or be derived from a mammalian, yeast or insect source. The vector used for homologous recombination may alternatively be designed such that the endogenous gene mutates or is modified in some other way during homologous recombination but still encodes the functional protein (for example, the upstream regulatory region may have been modified in a way which modifies expression of the endogenous protein). The modified section of the gene of the invention is in the homologous recombination vector. The construction of suitable vectors for homologous recombination is described, for example, in Thomas, K. R. and Capecchi, M. R. (1987) Cell 51: 503.

Any prokaryotic or eukaryotic organisms are in principle suitable for being used as recombinant host organisms for the nucleic acid of the invention or to the nucleic acid construct. Advantageously used host organisms are microorganisms such as bacteria, fungi or yeasts. Gram-positive or Gram-negative bacteria, preferably bacteria of the families Enterobacteriaceae, Pseudomonadaceae, Rhizobiaceae, Streptomycetaceae or Nocardiaceae, particularly preferably bacteria of the genera Escherichia, Pseudomonas, Streptomyces, Nocardia, Burkholderia, Salmonella, Agrobacterium or Rhodococcus, are advantageously used.

Depending on the host organism, the organisms used in the method of the invention are grown or cultured in a manner known to the skilled worker. Microorganisms are usually grown in a liquid medium comprising a carbon source usually in the form of sugars, a nitrogen source usually in the form of organic nitrogen sources such as yeast extract or salts such as ammonium sulfate, trace elements such as iron, manganese, magnesium salts, and, if appropriate, vitamins, at temperatures of between 0 and 100° C., preferably between 10 and 60° C., while being gassed with oxygen. The pH of the nutrient liquid may or may not be maintained here at a fixed value, i.e. regulated during growth. Growth may take place batch-wise, semibatch-wise or continuously. Nutrients may be introduced initially at the beginning of the fermentation or be subsequently fed in semicontinuously or continuously. The enzymes may be isolated from the organisms using the method described in the examples or be used for the reaction as a crude extract.

The invention furthermore relates to methods of recombinantly producing polypeptides of the invention or functional, biologically active fragments thereof, which methods comprise culturing a polypeptide-producing microorganism, if appropriate inducing expression of said polypeptides and isolating them from the culture. In this way the polypeptides may also be produced on an industrial scale if desired. The recombinant microorganism may be cultured and fermented by known methods. For example, bacteria can be propagated in TB medium or LB medium and at a temperature of from 20 to 40° C. and a pH of from 6 to 9. Suitable culturing conditions are described in detail in, for example, T. Maniatis, E. F. Fritsch and J. Sambrook, Molecular Cloning: A Laboratory Manual, Cold Spring Harbor Laboratory, Cold Spring Harbor, N.Y. (1989).

If the polypeptides are not secreted into the culture medium, the cells are then disrupted and the product is isolated from the lysate by known methods of isolating proteins. The cells may optionally be disrupted by high-frequency ultrasound, by high pressure, for example in a French pressure cell, by osmolysis, by the action of detergents, lytic enzymes or organic solvents, by using homogenizers or by a combination of several of the methods listed.

The polypeptides may be purified by means of known, chromatographic methods such as molecular sieve chromatography (gel filtration), such as Q-Sepharose chromatography, ion exchange chromatography and hydrophobic chromatography, and also by means of other customary methods such as ultrafiltration, crystallization, salting out, dialysis and native gel electrophoresis. Suitable methods are described, for example, in Cooper, F. G., Biochemische Arbeitsmethoden [original title: The tools of biochemistry], Verlag Water de Gruyter, Berlin, New York or in Scopes, R., Protein Purification, Springer Verlag, New York, Heidelberg, Berlin.

Another embodiment of the invention are the novel HABPs according to Formula I:

a₀₋₁-b₀₋₁₀-y₁₋₁₀-c₀₋₁₀-d₀₋₁ (I)

wherein
a,d stand independently from each other for a hydrophobin or an antifreeze protein sequence or an oral care active ingredient
b,c stand for independently from each other for an amino acid
y stands for a polypeptide sequence depicted in SEQ ID NO: 1 until SEQ ID NO:61 Preferred HABPs are such where y stands for SEQ ID NO:1,2,3,4.

SEQ ID NO: 1
IDDYTRA
SEQ ID NO: 2
HPPLHHA
SEQ ID NO: 3
SPSTHWK
SEQ ID NO: 4
GSPATAA
SEQ ID NO: 5
GKVQAQS
SEQ ID NO: 6
YPVTPSI
SEQ ID NO: 7
IPTLPSS
SEQ ID NO: 8
YQGASEN
SEQ ID NO: 9
EHITTSN
SEQ ID NO: 10
RILITIP
SEQ ID NO: 11
IPITNLR
SEQ ID NO: 12
TTSTRHI
SEQ ID NO: 13
NERALTL
SEQ ID NO: 14
MQTVEVT
SEQ ID NO: 15
SWGTQTD
SEQ ID NO: 16
TLPASSV

In some embodiments, the HABP is present in the composition in an amount of from 0.01 weight % to 3 weight % by total weight of the composition. In some embodiments, the HABP is present in the composition in an amount of from 0.1 weight % to 3 weight %, or from 0.1 weight % to 2 weight %, or from 0.1 weight % to 1 weight % by total weight of the composition. In other embodiments, the HABP is present in the composition in an amount of from 0.05 weight % to 1 weight %, or from 0.1 weight % to 0.5 by total weight of the composition In further embodiments, the HABP is present in the composition in an amount of from 0.5 weight % to 3 weight %, or from 0.5 weight % to 2 weight %, or from 0.5 weight % to 1 weight % by total weight of the composition. In still further embodiments, the HABP is present in the composition in an amount of from 1 weight % to 3 weight %, or from 1 weight % to 2 weight % by total weight of the composition.

Oral care active ingredients may be coupled in the form of “HBAP peptide—oral care active ingredient-hybrid molecules” to the described HBAP peptides. Preferably, “HBAP peptide—cooling compound hybrid molecules” are formed. A person skilled in the art is familiar with ways of coupling of oral care active ingredients such as cooling compounds to HBAP peptides. Suitability of individual methods depends on the availability of functional groups on the oral care active ingredient, preferably the cooling compound. Active groups on the HBAP peptides include amino, carboxyl, hydroxyl, and thiol groups. Preferred is a coupling via the terminal amino group or the terminal carboxyl group of the HBAP peptide.

In one embodiment, an oral care active ingredient, preferably a cooling compound, is coupled directly to the HBAP peptides.

In one embodiment, an oral care active ingredient, preferably a cooling compound, is coupled to the HBAP peptides via a bifunctional linker/crosslinker/spacer. Various molecules are available and a person skilled in the art is able to select the most suitable one.

Crosslinkers may be selected from homobifunctional and heterobifunctional crosslinkers. Homobifunctional crosslinkers are those having two identical reactive groups and often are used in one-step reaction procedures to crosslink proteins/peptides/oral care active ingredients to each other.

Heterobifunctional crosslinkers according to the invention possess two different reactive groups that allow for sequential (two-stage) conjugations, helping to minimize undesirable polymerization or self-conjugation. Potential moieties such as amine, sulfhydryls, carboxyls, phenols and carbohydrates may be targets of the reaction. This two-step strategy allows the modification of molecules having different accessible groups. Non-limiting example of suitable crosslinkers include those which are amine-reactive at one end and sulfhydryl-reactive at the other end. In sequential procedures, heterobifunctional reagents may be reacted with one reaction partner using e.g. the most reactive group of the crosslinker first. After removing excess of unreacted crosslinker, the modified first reaction partner may be added to a solution containing the second reaction partner where reaction through the second reactive group of the crosslinker can occur.

Suitable heterobifunctional crosslinkers include those having an amine-reactive succinimidyl ester (e.g., NHS-ester) at one end and a sulfhydryl-reactive group on the other end. The sulfhydryl-reactive groups are usually maleimides, pyridyl disulfides and α-haloacetyls. The NHS-ester reactivity may be less stable in aqueous solution and therefore may react first in sequential crosslinking procedures. NHS-esters may react with amines to form amide bonds.

Carbodiimides are zero-length crosslinkers (e.g., EDC) and may affect direct coupling between carboxylates (—COOH) and primary amines (—NH2). Other suitable heterobifunctional reagents may have one reactive group that is photoreactive rather than thermoreactive. This reactivity may allow for specific attachment of the thermoreactive groups first; subsequently, conjugation to any adjacent N—H or C—H sites may be initiated through the photoreactive group by activation with UV light. The reactivity of the photochemical reagent may allow for formation of a conjugate that may not be possible with a group-specific reagent. SFAD (sulfosuccinimidyl(perfluoroazidobenzamido=ethyl-1,3′-dithiopropionate) is an example of a photoactivatable reagent that contains a perfluorophenyl azide with an insertion efficiency of about 70%.

Further suitable linkers/spacers/crosslinkers are commercially available (e.g. on the web site—www.thermo.com/pierce—a crosslinker selection guide is listed with relevant parameters and features “Crosslinking Technical Handbook”). Non-limiting examples include reactive crosslinker groups and their functional group targets and/or amine/carboxyl, amine/sulfhydryl, sulfo-SMCC amine/sulfhydryl, MBS amine/sulfhydryl, sulfo-MBS amine/sulfhydryl, SMPB amine/sulfhydryl, sulfo-SMPB amine/sulfhydryl, GMBS amine/sulfhydryl, and sulfo-GMBS amine/sulfhydryl. A person skilled in the art is familiar with selecting suitable compounds and corresponding coupling reactions.

A suitable crosslinker include poly (ethylene glycol) (PEG) because it is known to be generally inert and provides a hydrophilic spacer between the peptide and the active molecule of interest. The length of the spacer chain of the linker can vary depending on the size of the peptide or other molecule that is attached at the distal end of the linker. However, a length of one to twenty ethylene glycol units is preferred.

Suitable linker/spacer/crosslinker include bicarboxylic acids (e.g. oxalic acid, malonic acid, succinic acid, glutaric acid, adipic acid, pimelic acid, suberic acid, azelaic acid, sebacic acid decanedioic acid, brassilic acid, thapsic acid) and diamines (e.g. ethylenediamine and related derivatives include the N-alkylated compounds, 1,1-dimethylethylenediamine, 1,1-dimethylethylenediamine, ethambutol and TMEDA; 1,3-diaminopropane; putrescine (butane-1,4-diamine); cadaverine (pentane-1,5-diamine); hexamethylenediamine; phenylenediamines; 2,5-Diaminotoluene; dimethyl-4-phenylenediamine, N,N′-di-2-butyl-1,4-phenylenediamine; 4,4′-diaminobiphenyl; 1,8-diaminonaphthalene).

Cooling compounds to be bound to peptides via crosslinkers as described include but are not limited to menthol and menthol derivatives (e.g. L-menthol, D-menthol, racemic menthol, isomenthol, neoisomenthol, neomenthol), menthone, menthone glycerine acetal (Frescolate® MGA), mono-menthyl succinate (Physcool®), mono-menthyl glutarate, 0-menthyl glycerine, or menthyl-N, N-dimethylsuccinamate) menthyl ether (e.g. (1-menthoxy)-1,2-propanediol, (1-menthoxy)-2-methyl-1,2-propanediol, 1-menthyl-methyl ether), menthyl ester (e.g. menthyl formiate, menthyl acetate, menthyl isobutyrate, menthyl lactates, L-menthyl-L-lactate, L-menthyl-D-lactate, menthyl-(2-methoxy)acetate, menthyl-(2-methoxy ethoxy)acetate, menthyl pyroglutamate), menthyl carbonates (e.g. menthyl propylene glycol carbonate, menthyl ethylene glycol carbonate, menthyl glycerine carbonate or mixtures thereof), the semi-esters of menthols with a dicarboxylic acid or their derivatives (e.g. mono-menthyl succinate, mono-menthyl glutarate, mono-menthyl malonate, 0-menthyl succinic acid ester-N,N-(dimethyl)amide, 0-menthyl succinic acid ester amide), menthane carboxylic acid amide (e.g. menthane carboxylic acid-N-ethylamid [W53], Na-(menthane-carbonyl)glycine ethyl ester [WS5], menthane carboxylic acid-N-(4-cyanophenyl)amide, menthane carboxylic acid-N-(alkoxyalkyl)amide), menthone and menthone derivatives (e.g. L-menthone glycerine ketal), 2,3-dimethyl-2-(2-propyl)-butyric acid derivatives (e.g. 2,3-dimethyl-2-(2-propyl)-butyric acid-N-methyl amide [WS23]), isopulegol or its esters (1-(−)-isopulegol, 1-)-isopulegol acetate), menthane derivatives (e.g. p-menthane-3,8-diol), cubebol or synthetic or natural mixtures containing cubebol, pyrrolidone derivates of cycloalkyl dione derivatives (e.g. 3-methyl-2(1-pyrrolidinyl)-2-cyclopentene-1-one) or tetrahydropyrimidine-2-ones (e.g. Icilin or related compounds such as those described in WO 2004/026840). Further examples may be disclosed in US 20150086491 A1 and are deemed to be suitable cooling compounds herein. Preferred are menthol and menthol derivatives, such as 5-methyl-2-(propan-2-yl) cyclohexane-1-carboxylic acid.

Another embodiment of the invention is a method of binding or targeting to the surface of enamel and/or dentin, comprising applying to the teeth an oral care composition comprising:
a. an HABP;
b. an orally acceptable carrier,
   wherein HABP is present in the composition in an amount of from 0.01 weight % to 3 weight % by total weight of the composition.
      Another embodiment of the invention is a method of repairing or inhibiting dental erosion, comprising applying to the teeth an oral care composition:
a. an HABP;
b. an orally acceptable carrier,
   wherein HABP is present in the composition in an amount of from 0.01 weight % to 3 weight % by total weight of the composition.

In one embodiment, the binding of “HBAP peptide—oral care active ingredient-hybrid molecules” to the surface of enamel is non-covalent by nature. Preferably, cooling compounds are non-covalently bound via HABP to enamel. The cooling compound may consequently be released over time from the enamel surface by mechanical forces and/or enzymatic cleavage and/or spontaneous drop-off. A slow release form of the cooling compound may result in a long lasting cooling effect due to a different kinetic when compared to a cooling compound not bound to HABP.

Another embodiment of the invention is a method of promoting dental remineralization, and/or enhancing the anti-cavity effects of fluoride comprising applying to the teeth an oral care composition comprising:
a. an HABP;
b. an orally acceptable carrier,
   wherein HABP is present in the composition in an amount of from 0.01 weight % to 3 weight % by total weight of the composition.

The expression “orally acceptable carrier” as used herein denotes a carrier made from materials that are safe and acceptable for oral use in the amounts and concentrations intended, for example materials as would be found in conventional toothpaste and mouthwash. Such materials include water or other solvents that may contain a humectant such as glycerin, sorbitol, xylitol and the like. In some aspects, the term “orally acceptable carrier” encompasses all of the components of the oral care composition except for the hydrolyzed plant protein and the fluoride. In other aspects, the term refers to inert or inactive ingredients that serve to deliver the hydrolyzed plant protein, and/or any other functional ingredients, to the oral cavity.

Orally acceptable carriers for use in the invention include conventional and known carriers used in making mouth rinses or mouthwashes, toothpastes, tooth gels, tooth powder, lozenges, gums, beads, edible strips, tablets and the like. Carriers should be selected for compatibility with each other and with other ingredients of the composition.

The following non-limiting examples are provided. In a toothpaste composition, the carrier is typically a water/humectant system that provides a major fraction by weight of the composition. Alternatively, the carrier component of a toothpaste composition may comprise water, one or more humectants, and other functional components other than the hydrolyzed wheat protein or hydrolyzed rice protein. In a mouth rinse or a mouthwash formulation, the carrier is typically a water/alcohol liquid mixture in which the hydrolyzed wheat protein or hydrolyzed rice protein is dissolved or dispersed. In a dissolvable lozenge, the carrier typically comprises a solid matrix material that dissolves slowly in the oral cavity. In chewing gums, the carrier typically comprises a gum base, while in an edible strip, the carrier typically comprises one or more film forming polymers.

The oral care compositions provided herein may further comprise one or more additional ingredients selected from abrasives, pH modifying agents, surfactants, foam modulators, thickening agents, viscosity modifiers, humectants, anti-calculus or tartar control agents, sweeteners, flavorants, colorants and preservatives. These ingredients may also be regarded as carrier materials. Non-limiting examples are provided below.

In one embodiment a composition of the invention comprises at least one abrasive, useful, for example, as a polishing agent. Any orally acceptable abrasive can be used, but the type, fineness (particle size) and amount of abrasive should be selected so that tooth enamel is not excessively abraded during normal use of the composition. Suitable abrasives include, without limitation, silica, for example in the form of silica gel, hydrated silica or precipitated silica, alumina, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, [beta]-calcium pyrophosphate, tricalcium phosphate, calcium polymetaphosphate and insoluble sodium polymetaphosphate. One or more abrasives are optionally present in the oral care compositions of the present invention in an amount of 1 weight % to 5 weight % by total weight of the composition. The average particle size of an abrasive, if present, is generally 0.1 to 30 μm, and preferably, 5 to 15 μm.

In a further embodiment an oral care composition of the invention comprises at least one bicarbonate salt, useful, for example, to impart a “clean feel” to teeth and gums due to effervescence and release of carbon dioxide. Any orally acceptable bicarbonate can be used, including, without limitation, alkali metal bicarbonates such as sodium and potassium bicarbonates, ammonium bicarbonate and the like. One or more bicarbonate salts are optionally present in a total amount of 1 weight % to 10% by weight of the composition.

In a still further embodiment, an oral care composition of the invention comprises at least one pH modifying agent. Such agents include acidifying agents to lower pH, basifying agents to raise pH and buffering agents to control pH within a desired range. For example, one or more compounds selected from acidifying, basifying and buffering agents can be included to provide a pH of 2 to 10, or in various illustrative embodiments a pH of 2 to 8, 3 to 9, 4 to 8, 5 to 7, 6 to 10, or 7 to 9. Any orally acceptable pH modifying agent can be used, including, without limitation, carboxylic, phosphoric and sulfonic acids, acid salts (for example, monosodium citrate, disodium citrate, monosodium malate), alkali metal hydroxides such as sodium hydroxide, carbonates such as sodium carbonate, bicarbonates, borates, silicates, phosphates (for example, monosodium phosphate, trisodium phosphate, pyrophosphate salts) imidazole and the like. One or more pH modifying agents are optionally present in a total amount effective to maintain the composition in an orally acceptable pH range.

In a still further embodiment a composition of the invention comprises at least one surfactant, useful, for example, to provide enhanced stability to the composition and the components contained therein, to aid in cleaning a dental surface through detergent action, and to provide foam upon agitation (for example, during brushing with a dentifrice composition of the invention). Any orally acceptable surfactant, including those which are anionic, nonionic or amphoteric, can be used. Suitable anionic surfactants include, without limitation, water-soluble salts of C₈₋₂₀ alkyl sulfates, sulfonated monoglycerides of C₈₋₂₀ fatty acids, sarcosinates, taurates and the like. Suitable nonionic surfactants include, without limitation, poloxamers, polyoxyethylene sorbitan esters, fatty alcohol ethoxylates, alkylphenol ethoxylates, tertiary amine, oxides, tertiary phosphine oxides, dialkyl sulfoxides and the like. Suitable amphoteric surfactants, without limitation, derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of 0.01 weight % to 10 weight %, for example, from 0.05 weight % to 5 weight % or from 0.1 weight % to 2 weight % by total weight of the composition.

In a still further embodiment, an oral care composition of the invention comprises at least one foam modulator, useful, for example, to increase the amount, thickness or stability of foam generated by the composition upon agitation. Any orally acceptable foam modulator can be used including, without limitation, polyethylene glycols (PEGs). One or more PEGs are optionally present in a total amount of from 0.1 weight % to 10 weight by total weight of the composition.

In a still further embodiment, an oral care composition of the invention comprises at least one thickening agent, useful, for example, to impart a desired consistency and/or mouth feel to the composition. Any orally acceptable thickening agent can be used including, without limitation, carbomers (carboxyvinyl polymers), carrageenans, cellulosic polymers such as hydroxyethylcellulose, carboxymethylcellulose (CMC) and salts thereof, natural gums such as karaya, xanthan, gum arabic and tragacanth, colloidal magnesium aluminum silicate, colloidal silica and the like. One or more thickening agents are optionally present in a total amount of 0.01 weight % to 15 weight %, by total weight of the composition.

In a still further embodiment a composition of the invention comprises at least one viscosity modifier, useful, for example, to inhibit settling or separation of ingredients or to promote re-dispersion of ingredients upon agitation of a liquid composition. Any orally acceptable viscosity modifier can be used including, without limitation, mineral oil, petrolatum, clays, silica and the like. One or more viscosity modifiers are optionally present in a total amount of 0.01 weight % to 10 weight %, by total weight of the composition.

In a still further embodiment, an oral care composition of the invention comprises at least one humectant which may be used to prevent hardening of a toothpaste upon exposure to air. Any orally acceptable humectant can be used, including, without limitation, polyhydric alcohols such as glycerin, sorbitol, xylitol or low molecular weight PEGs. Most humectants also function as sweeteners. One or more humectants are optionally present in a total amount of 1 weight % to 50 weight % by total weight of the composition.

In a still further embodiment, an oral care composition of the invention comprises at least one sweetener which enhances taste of the composition. Any orally acceptable natural or artificial sweetener can be used including, without limitation, dextrose, sucrose, maltose, dextrin, mannose, xylose, ribose, fructose, levulose, galactose, corn syrup, partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount of 0.005 weight % to 5 weight % by total weight of the composition.

In a still further embodiment, an oral care composition of the invention comprises at least one flavorant which enhances the taste of the composition. Any orally acceptable natural or synthetic flavorant can be used including, without limitation, vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences, and the like. Also encompassed within flavorants are ingredients that provide fragrance and/or other sensory effects in the mouth, including cooling or warming effects. Such ingredients illustratively include menthol, menthyl acetate, menthyl lactate, camphor, eucalyptus oil, eucalyptol, eugenol, cassia, oxanone, α-irisone, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of 0.01 weight % to 5 weight %, by total weight of the composition.

In a still further embodiment, an oral care composition of the invention comprises at least one colorant. A colorant can serve a number of functions. These include providing a white or light-colored coating on a dental surface, indicating locations on a dental surface that have been effectively contacted by the composition, and/or modifying the appearance of the composition to enhance attractiveness to the consumer. Any orally acceptable colorant can be used including, without limitation, talc, mica, magnesium carbonate, calcium carbonate, magnesium silicate, magnesium aluminum silicate, silica, titanium dioxide, zinc oxide, iron oxide, ferric ammonium ferrocyanide, manganese violet, titaniated mica, bismuth oxychloride and the like. One or more colorants are optionally present in a total amount of 0.001 weight % to 20 weight % by total weight of the composition.

In a still further embodiment, an oral care composition of the invention comprises a preservative. The preservative may be selected from parabens, potassium sorbate, benzyl alcohol, phenoxyethanol, polyaminopropryl biguanide, caprylic acid, sodium benzoate and cetylpyridinium chloride. In some embodiments, the preservative is present at a concentration of from about 0.001 to about 1 weight %, by total weight of the composition.

In a still further embodiment, an oral care composition of the invention is a chewing gum comprising gum base, flavor, sweetening agent and HABP. The gum base is present from about 4.8% to about 90%, the flavor from about 0.1% to about 10%, the sweetening agent from about 0.1% to about 95% and the HABP from about 0.01% to about 0.5%.

The following examples illustrate compositions of the invention and their uses. The exemplified compositions are illustrative and do not limit the scope of the invention.

### EXAMPLES

### Example 1—Binding Assay

i. Petide/Protein Labeling
For binding studies 5(6)-carboxyfluorescein was conjugated to the N-terminus of the peptides/proteins by using the corresponding succinimidyl ester (Invitrogen, Darmstadt, Germany). The labeling reaction was performed according to the manufactures protocol (Invitrogen, Darmstadt, Germany). After conjugation, the labeled peptide/protein was purified either with a PD-10 desalting column for proteins larger than 5 kDa or a PD MidiTrap G-10 for smaller proteins (GE Healthcare, Buckinghamshire, UK) or for peptides by HPLC on RP18 column.
ii. Peptide/Protein Binding
For the binding experiments N-terminal labeled peptides/proteins were used in a final concentration of 2 μM. The binding was carried out in HBS-T buffer (150 mM NaCl, 50 mM HEPES pH 7.5, 0.1% Tween 20). The peptides/proteins were exposed to 10 mg hydroxyapatite powder (Fluke) for 2 h at 37° C. After five wash steps with HBS-T buffer the bound peptide/protein was visualized by fluorescence microscopy with the appropriate filters (excitation 475/50; emission 525/50). To quantify the binding, the fluorescence intensity of the solution before (Fi) and after (Ff) binding was measured with a Fluostar Galaxy (BMG, Ortenberg, Germany) and the amount of bound peptide/protein was estimated (cbound=[1−(Ff/Fi)]×c0). The peptide LIKHILHRL, which was described as a non-binding peptide to hydroxyapatite, was used as a negative control [Yarbrough D K, Hagerman E, Eckert R, He J, Choi H, Cao N, et al. Specific binding and mineralization of calcified surfaces by small peptides. Calcif Tissue Int 2010; 86(1):58-66.]. All binding experiments were carried out as triplicates.
Evaluation: +: 30-50% bound peptide on HAP; ++: 50-100% bound peptide on HAP

| SEQ ID NO: 1 | IDDYTRA | ++ |
|---|---|---|
| SEQ ID NO: 2 | HPPLHHA | + |
| SEQ ID NO: 3 | SPSTHWK | + |
| SEQ ID NO: 4 | GSPATAA | + |
| SEQ ID NO: 5 | GKVQAQS | + |
| SEQ ID NO: 6 | YPVTPSI | + |
| SEQ ID NO: 7 | IPTLPSS | ++ |
| SEQ ID NO: 8 | YQGASEN | ++ |
| SEQ ID NO: 9 | EHITTSN | + |
| SEQ ID NO: 10 | RILITIP | + |
| SEQ ID NO: 11 | IPITNLR | + |
| SEQ ID NO: 12 | TTSTRHI | + |
| SEQ ID NO: 13 | NERALTL | + |
| SEQ ID NO: 14 | MQTVEVT | + |
| SEQ ID NO: 15 | SWGTQTD | + |
| SEQ ID NO: 16 | TLPASSV | ++ |
| SEQ ID NO: 17 | AFP4-His6 | + |
| | | (35%) |
| SEQ ID NO: 18 | IPTLPSS-GGG-AFP4-His6 | ++ |
| | | (60%) |

### Example 2—Hydroxyapatite Nucleation Assay

To determine the anti-erosion/HAP-nucleating abilities of the unlabeled peptides/proteins the decrease in Ca²⁺ concentration was measured. Nucleation was carried out in artificial saliva [Panich M, Poolthong S. The effect of casein phosphopeptide-amorphous calcium phosphate and a cola soft drink on in vitro enamel hardness. J Am Dent Assoc 2009; 140:455-60.]. Briefly, one 10× stock solution of 46.2 mM K₂HPO₄, 26.8 mM KH₂PO₄ and a second of 87.2 mM KCl, 6.1 mM MgCl₂, 14.9 mM CaCl₂) were prepared. For the nucleation experiments, artificial saliva containing 8.7 mM KCl, 0.6 mM MgCl₂, 1.5 mM CaCl₂), 4.6 mM K₂HPO₄, 2.7 mM KH₂PO₄ and 25 μM peptide/protein was prepared in a final volume of 1 mL. As a negative control only the nucleation solution without any peptide/protein was used. All solutions were filtered (0.2 μm) prior to the use to avoid uncontrolled nucleation due to small particles. Periodically, 30 μL samples were taken every hour for a total of 5 h, centrifuged 2 min, 13000 rpm and the calcium concentration of the supernatant was determined. For the calcium detection, 30 μM o-cresolphthalein, 2.7 mM 8-hydroxyquinoline, 20 mM 2-amino-2-methyl-1-propanol pH 10.5 and 20 μL of the sample were mixed and the absorbance was monitored at 575 nm using a Spectramax (Molecular Devices, Biberach, Germany). All nucleation experiments were carried out at 37° C. and as triplicates. Addition of unlabeled peptides (seq no. 1-no. 16) resulted in increased nucleation by a factor of 1.3-2.

### Example 3—HABP Peptide—Cooling Compounds Hybrid Molecules

Peptides according to SEQ ID NO: 1 to 16 can be coupled to cooling compounds by using e.g. oxalate (formula A), glutarate (formula B), or succinate (formula C) as crosslinker:

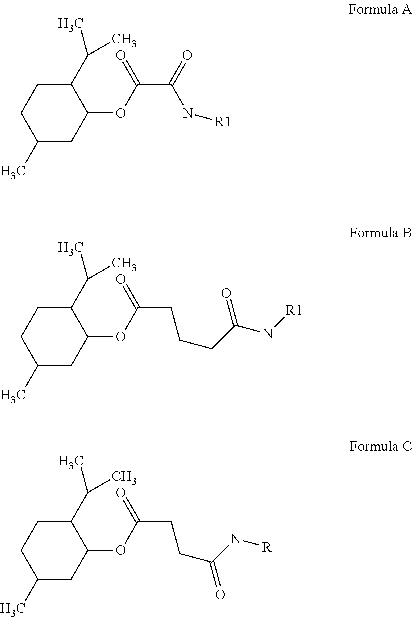

### Example 4—HABP Peptide—Menthol Hybrid Molecule

Coupling of 5-methyl-2-(propan-2-yl) cyclohexane-1-carboxylic acid was performed at the N-terminus of a resin-bound HBAP peptides according to SEQ ID Nos: 6, 7, 8, and 16 to form the molecule according to formula D:

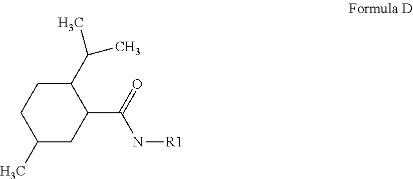

### Example 5—Release of HABP Peptide—Menthol Hybrid Molecules from Calcium Phosphate Discs

The hybrid molecules of example 4 (5 μM in Tris buffer pH 8) were incubated with calcium phosphate discs (HA-discs from Himed) at 37° C. for 1 hr. Unbound molecules were removed by washing 3-times with Tris buffer.
The coated calcium phosphate discs were incubated with buffer as a control and in parallel with pooled human saliva (5 donors; 37° C., for 2 hrs). The supernatant was centrifuged and evaluated for cooling activity by specific activation of human cool receptor TRPM8.

### Example 6—Incubation of HABP Peptide—Menthol Hybrid Molecules from Calcium Phosphate Discs (HA-Discs)

The HABP peptide—cooling compound-hybrid molecules of example 4 (1 μM in Tris buffer pH 8) were incubated with pooled human saliva (5 donors) 1:1 or as reference 1:1 in Tris buffer pH 8; 37° C., for 2 hrs at 37° C. Samples were frozen until evaluation in a cell based assay for activation of the human TRPM8 receptor.

### Example 7—Assay on TRPM8 Activation

A test comparable with that previously described in the literature by Behrendt H. J. et al., Br. J. Pharmacol. 141, 2004, 737-745, was carried out (see also US 20150086491 A1). The agonisation or antagonisation of the receptor was quantified by means of a Ca²⁺-sensitive dye (e.g. FURA, Fluo-4, etc.). Agonists on their own bring about an increase in the Ca²⁺-signal; antagonists in the presence of, for example, menthol bring about a reduction in the Ca²⁺-signal (in each case detected by means of the Fluo-4 dye, which due to the Ca²⁺ has other fluorescent properties). To begin with, in a manner known per se, in cell culture flasks a fresh culture of transformed HEK cells was prepared. The HEK293-TRPM8 test cells were removed using trypsin from the cell culture flasks and 40 000 cells/well were sown with 100 μl medium in 96-well plates (Greiner #655948 Poly-D-lysine coated). In order to induce the TRPM8 receptor the growth medium tetracycline was mixed in (DMEM/HG, 10% FCS tetracycline-free, 4 mM L-glutamine, 15 μg/ml blasticidin, 100 μg/ml hygromycin B, 1 μg/ml tetracycline). The next day the cells were charged with Fluo-4 dye (non-fluorescent acetoxymethyl ester Fluo-4 AM; 2,2′-((2-(2-(2-(bis(carboxymethyl)amino)-5-(2,7-difluoro-6-hydroxy-3-oxo-3H-xanthen-9-yl)phenoxy)ethoxy)-4-methylphenyl)azanediyl)diacetic acid) and the test was performed. The procedure was as follows: Addition of 100 μl/well of dye solution Ca-4 Kit (RB 141, Molecular Devices) per 100 μl of medium (DMEM/HG, 10% FCS tetracycline-free, 4 mM L-glutamine, 15 μg/ml blasticidin, 100 μg/ml hygromycin B, 1 μg/ml tetracycline).
Incubation in the incubator for 30 minutes/37° C./5% CO₂, 30 minutes/20° C.
Preparation of the test samples=supernatant of calcium phosphate discs or peptide-active-ingredient-hybrid+saliva incubation in triplicate (different dilution of samples: 1/5/10 μl in 200 μl HBSS buffer), and of positive controls (different concentrations of menthol or icilin or ionomycin in 200 μl HBSS buffer) and negative controls (just 200 μl of HBSS buffer).
Addition of the test samples in quantities of 50 μl/well and measurement of the change in fluorescence (e.g. in the FLIPR assay device, Molecular Devices or NovoStar, BMG) at 485 nm excitation, 520 nm emission, and evaluation of the effective strength of the various substances/concentrations and determination of the EC50 values.
Results of evaluation on TRPM8 receptor (cooling sensation):

| | menthol-peptide hybrid molecule class | | | | | |
|---|---|---|---|---|---|---|
| | II | III | II | III | II | III |
| peptide | no saliva incubation (buffer | | after saliva incubation in | | supernatant of HA-discs | |
| sequence | control) in solution | | solution | | after saliva incubation | |
| #6 | + | ++ | ++ | +++ | + | ++ |
| #7 | + | + | + | +++ | + | ++ |
| #8 | − | − | + | + | − | + |
| #16 | − | + | − | + | − | + |
| positive | +++ | +++ | +++ | +++ | − | − |
| reference | | | | | | |
| menthol (no | | | | | | |
| peptide) | | | | | | |
| negative | − | − | − | − | − | − |
| control | | | | | | |
| (buffer) | | | | | | |

The evaluation showed that the reference molecule menthol is bioactive on the human cool receptor TRPM8 (before and after incubation with saliva), but nothing is bound on hydroxyapatite. The peptide modification of menthol resulted in a change of property: the hybrid molecule was bound on hydroxyapatite and released as a cooling active ingredient after incubation with human saliva.

While particular embodiments of the invention have been illustrated and described, it will be obvious to those skilled in the art that various changes and modifications may be made without departing from the scope of the invention as defined in the appended claims.

1. A method of binding or targeting to the surface of enamel and/or dentin, repairing or inhibiting dental erosion, promoting dental remineralization, and/or enhancing the anti-cavity effects of fluoride comprising applying to the teeth an oral care composition comprising and/or enhancing the biological activity of active ingredients by increasing the local concentration on the surface of enamel/dentin:
a) an HABP;
b) an orally acceptable carrier,
wherein HABP is present in the composition in an amount of from 0.01 weight % to 3 weight % by total weight of the composition.

2. The method of claim 1 wherein HABP has the polypeptide sequence depicted in Formula (I)
a₀₋₁-b₀₋₁₀-y₁₋₁₀-c₀₋₁₀-d₀₋₁ (I)
wherein
a,d stand for independently from each other a hydrophobin or an antifreeze protein sequence or an oral care active ingredient,
b,c stand for independently from each other for an amino acid
y stands for a polypeptide sequence depicted in SEQ ID NO: 1 until SEQ ID NO:16.

3. The method of claim 2 wherein Formula I has the following meaning:
a,d=0
b,c=0
y₁=SEQ ID NO: 1, 2, 3, 4.

4. The method of claim 1 wherein HABP has been neutralized to approximately neutral or slightly basic pH.

5. The method described in claim 1 wherein the oral care composition further comprises an effective amount of fluoride.

6. The method of claim 1 wherein HABP is present in the composition in an amount of from 0.1 weight % to 3 weight % by total weight of the composition.

7. The method of claim 1 wherein the amount of fluoride is about 500 ppm fluoride.

8. The method of claim 1 wherein the composition is a dentifrice.

9. The method of claim 1 which is a method for reducing, inhibiting or repairing dental erosion.

10. The method of claim 1 which is a method for promoting dental remineralization.

11. The method of claim 1 which is a method for enhancing the anti-cavity effects of fluoride.

12. A dentifrice comprising
a) HABP, which has been neutralized to approximately neutral or slightly basic pH;
b) an effective amount of fluoride and
c) an orally acceptable carrier.

13. An oral care composition comprising:
a) HABP;
b) optionally an effective amount of fluoride;
c) an orally acceptable carrier,
wherein HABP is present in the composition in an amount of from 0.01 weight % to 3 weight % by total weight of the composition, for use in repairing or inhibiting dental enamel erosion, promoting remineralization of dental enamel, and/or enhancing the anti-cavity effects of fluoride.

14. (canceled)

15. A method of making an oral care product comprising HABP comprising
a) neutralizing a HABP solution by dilution with an aqueous buffer solution to obtain a solution having approximately neutral or slightly basic pH;
b) filtering and centrifuging the solution product of a) to obtain a filtrate comprising HABP;
c) adding fluoride and optionally a biocide to the filtrate product of b);
d) admixing the product of c) to components of an orally acceptable carrier.

16. An oral care composition comprising HABP, wherein the oral care composition is obtained or obtainable by the method of claim 15.

17. A hydroxyapatite binding polypeptide of the general Formula (I)
a₀₋₁-b₀₋₁₀-y₁₋₁₀-c₀₋₁₀-d₀₋₁ (I)
wherein
a,d stand independently from each other for a hydrophobin or an antifreeze protein sequence or an oral care active ingredient,
b,c stand for independently from each other for an amino acid
y stands for a polypeptide sequence depicted in SEQ ID NO: 1, 2, 3, 4.

18. A hydroxyapatite binding polypeptide of the general Formula (I)
a₀₋₁-b₀₋₁₀-y₁₋₁₀-c₀₋₁₀-d₀₋₁ (I)
wherein
a,d stand independently from each other for 0 or an oral care active ingredient,
b,c=0
y stands for a polypeptide sequence depicted in SEQ ID NO: 6, 7, 8, 16.

19. A chewing gum composition comprising
a) gum base,
b) flavor,
c) sweetening agent, and
d) about 0.01% to about 0.5% HABP by weight of the chewing gum composition.
